(19) United States
(12) Patent Application Publication
(10) Pub. No.: US 2023/0346555 A1
(43) Pub. Date: Nov. 2, 2023

(54) ANNULOPLASTY DEVICE

(71) Applicant: Vestlandets Innovasjonsselskap AS (VIS), Bergen (NO)

(72) Inventor: Vegard TUSETH, Bergen (NO)

(73) Assignee: Vestlandets Innovasjonsselskap AS (VIS), Bergen (NO)

(21) Appl. No.: 17/997,817

(22) PCT Filed: Jan. 20, 2022
(86) PCT No.: PCT/EP2022/051193

§ 371 Date: Nov. 2, 2022

(30) Foreign Application Priority Data
Jan. 20, 2021 (GB) 2100730.7

(51) Int. Cl. A61F 2/24

(52) U.S. Cl. CPC A61F 2/2445; A61F 2/2466; A61F 2220/0016; A61F 2230/0095

(57) ABSTRACT

An annuloplasty device for restoring a heart valve annulus from a dysfunctional configuration to a working configuration, wherein the device comprises a band which is expandable from a pre-set configuration, wherein the pre-set configuration of the band corresponds to the working configuration of the annulus.

The present invention relates to heart valve repair procedures and devices, and more particularly to transcatheter annuloplasty procedures and devices.

Annuloplasty is a procedure for the repair of heart valve, by tightening or strengthening the annulus around a valve. Annuloplasty procedures are often used to heart valve regurgitation. By way of example, the mitral valve regulates blood flow from the left atrium to the left ventricle, and prevents blood from flowing back into the left atrium from the left ventricle. Mitral valve regurgitation is a condition, which occurs when blood flows backwards into the left atrium across a so-called “leaky heart valve”. This backflow is caused by a dysfunctional heart valve, which may be dysfunctional through injury, malformation and/or disease.

If the condition is minor, no or little treatment is required. However, in some cases, the blood backflow places a strain on the heart and causes it to work harder to compensate for the leaked volumes. A conventional and effective surgical method for treating this condition is with the use of an annuloplasty band or ring, which reduces the size of the annulus around the leaky valve to restore normal working function.

In recent years, alternatives procedures, in particular using transcatheter technologies, have been developed to treat patients for whom surgical intervention is unsuitable. Transcatheter or percutaneous procedures are minimally invasive, and hence minimally traumatic.

A major challenge in transcatheter procedures is accessibility, including the delivery of medical tools and devices to the target site, and their precise and safe manipulation at the target site. Despite considerable advances in this field, it remains difficult to implant miniaturised devices in areas hindered by the patient's anatomy. Moreover, the medical practitioner must take into account the specific anatomical landscape (such as shape, dimensions, damage and condition) of the target area for a successful implantation.

Whilst in surgical procedures, an annuloplasty device of suitable shape and size can be directly manipulated, positioned and attached to the dysfunctional annulus, transcatheter implantation requires imaging equipment to visualise the device delivery path and the target site. There is also added complexity due to the accessibility of the target area, lack of direct visualisation and reduced size of the transcatheter tools and devices.

In transcatheter procedures, the annuloplasty device is delivered (and the medical tools manipulated) through a catheter, which conventionally travels through the patient's circulatory system. Due to the size restrictions, it is therefore difficult, if not impossible, to deliver an annuloplasty device with a fixed configuration. The fixed configuration would be the configuration of the device required to restore the annulus to a working configuration, and the lumen of the delivery catheter would need to be sufficiently large not only to accommodate this final configuration, but also to provide unhindered space for the surgeons to manipulates the tools and devices. Such a large catheter cannot travel through the patient's circulatory system. As a result, transcatheter annuloplasty devices are contractible or collapsible to enable delivery through a catheter.

Most established transcatheter annuloplasty procedures involve the use of a flexible or semi-flexible band or ring. Once at the target area, the band or ring is positioned by the surgeon, sutured, screwed or otherwise anchored to the degenerated annulus to form the desired final working configuration, and subsequently tightened to narrow the annulus.

Thus, it is a requirement for transcatheter annuloplasty devices to be collapsible in order to be delivered through a catheter, and a consequence of this requirement is that the surgeon must reconstruct in situ the working configuration of the annuloplasty device.

The heart valve anatomy is complex in itself, and repairing a heart valve with the currently available techniques is equally so. The development of new annuloplasty devices and of corresponding delivery tools, is costly, medical practitioners are required to undertake extensive and intensive training specific to each device-type before being able to fit them into patients. The process of establishing each new device in current medical practice is therefore highly resource-intensive.

Moreover, even with sophisticated devices and tools, intensive training and expert surgical intervention, there are so many variables at play that the outcome of the procedure is unpredictable. It is difficult to predict whether the band has been suitably positioned and anchored so that regurgitation may be significantly minimised, until the band has been tightened. At this stage, the device has already been sutured to the annulus, and can only be retrieved through surgery, if at all. It is also difficult to predict if the restoration of the valve is durable. In addition, repeated attempts at successful implantation and trial and error is damaging and traumatic to the patient's anatomy. Despite all efforts, the band may still fail to successfully restore the valve.

It is an object of this invention to mitigate problems such as those described above and to provide an improved alternative to existing products. In particular, there is a need for a simpler, more standardised, less resource-intensive and less traumatic procedure and device for repairing leaky heart valves.

According to a first aspect of the invention, there is provided an annuloplasty device for restoring a heart valve annulus from a dysfunctional configuration to a working configuration, wherein the device comprises a band which is expandable from a pre-set configuration, wherein the pre-set configuration of the band corresponds to the working configuration of the annulus.

Within the context of the invention, the annulus may have a “dysfunctional configuration” in which it has partially or completely lost its functional activity, namely, to provide structural support to the valve so that the valve allows flow from a first compartment to a second compartment and minimises backflow from the second compartment to the first compartment. A dysfunctional annulus may be (genetically or pathologically) degenerated and the terms are used herein interchangeably. The degeneration may for example originate from injury, malformation or injury, and often expresses itself as a loosening or dilatation of the annulus. By contrast, a “working configuration” is a configuration wherein the annulus provides adequate and sufficient structural support so as to minimise or avoid valve regurgitation.

The annuloplasty device according to the present invention comprises a band which is expandable from a pre-set configuration, which corresponds to the pre-set working configuration of the device and of the annulus.

An external force may be exerted to the device to expand from its pre-set working configuration to enable its anchoring to the dysfunctional annulus, and upon cessation of said force, the device automatically contracts or reverts back to its pre-set working configuration. This contraction mechanism is a key aspect of the present invention, in that it facilitates the simplification and standardisation of annuloplasty procedures. The present invention is advantageous over known transcatheter annuloplasty devices, which must be adjusted in situ, in the patient at the target site, by trained expert surgeons using remotely controlled tools and imaging equipment. By contrast, in the present invention, minimal intervention, estimation and adjustment by the surgeon are required, either before (e.g. during the grasping and securing phase) or after release of the device.

The pre-set configuration is one which restores a dysfunctional annulus into a working annulus. While sizes may differ from patient to patient, the overall average three-dimensional shape of working heart annuli is similar in patients and commonly known. The pre-set configuration may therefore translate into a three-dimensional shape suitable to restore the configuration of one or more sections of a dysfunctional annulus, and/or restore its overall configuration. The annuloplasty device is preferably manufactured with a pre-set configuration corresponding to the average three-dimensional shape of a working human heart valve for standardisation purposes, but may also be manufactured with modifications to treat specific defects and/or specific patient groups. The device may be supplied in different sizes.

Generally, “restoring” the annulus refers to the reduction of one or more dimensions of a pathological (large) annulus. Whilst the invention seeks to restore the annulus from a dysfunctional or degenerated configuration to a working configuration, it is envisaged to use the annuloplasty device and method in the context of preventative and palliative procedures, where it is desired to maintain or preserve one or more dimensions or shape of the annulus.

Within the context of the invention, “expandable” or “expanded” means that one or more dimensions of the expanded device is greater than the one or more corresponding dimensions of the device in the pre-set configuration. The expanded band has a configuration such that it can be secured to the degenerated or dilated annulus.

As used herein, the term “means” can be equivalently expressed as, or substituted with, any of the following terms: device, apparatus, structure, part, sub-part, assembly, sub-assembly, machine, mechanism, article, medium, material, appliance, equipment, system, body or similar wording.

The device according to the present invention does not require extensive manipulation and adjustment in situ. Once delivered to the target area, the device is positioned, and the band expanded to reach the attachment sites of the dilated annulus. Upon release, the expanded band collapses, contracts or reverts to its pre-set configuration, thereby automatically contracting the annulus into its working configuration to prevent significant amounts of blood flowing back through the heart valve.

In a preferred embodiment, the band is further compressible from the pre-set configuration. Within the context of the invention, “compressible” or “compressed” means that one or more dimensions of the compressed device is smaller than the one or more corresponding dimensions of the device in the pre-set configuration. The band may be deformable, foldable or collapsible, so as to achieve an expanded configuration and/or a compressed configuration. The compressed band has a configuration such that it can be fitted in the inner lumen of a delivery catheter or delivery sheath. Preferably, the compressed band is slidably fitted in the inner lumen of a delivery catheter or delivery sheath.

Transcatheter procedures are minimally invasive and allow patients to experience less discomfort and to recover in a shorter amount of time, compared to surgical procedures. However, transcatheter procedures involve smaller devices and instruments, which are more difficult to manipulate, and the surgeon's movement is further limited by hindrance and obstacles in the patient's own anatomy. Access is not an issue with surgical procedures, and there is therefore little need for additional complex insertion, delivery, viewing and implantation instruments. The device according to the present invention may be both expandable and compressible, and requires minimal manipulation in situ so that simple transcatheter instruments may be used.

The overall (or some) dimensions of the annulus in a working configuration are the same or smaller than the annulus in a degenerated configuration. Therefore, in the preferred embodiment, the annuloplasty device has a pre-set configuration with overall (or some) dimensions which are smaller than the dimensions of the expanded device in the deployed configuration; and/or the annuloplasty device has a pre-set configuration with overall (or some) dimensions which are greater than the dimensions of the collapsed or compressed device in the delivery configuration.

Preferably, the dimensions of the band in the expanded configuration are equal or greater than the dimensions of the degenerated annulus. Thus, the band can be expanded so as to reach the attachment sites of the annulus, and secure the band thereto.

Preferably, the band, in its pre-set configuration, is substantially rigid. The pre-set configuration of the band is a configuration which supports the annulus in a working configuration. Since the working configuration is the configuration in which the backwards flow is significantly minimised, there may be more than one working configuration of the annulus.

The band in its pre-set configuration may be sufficiently rigid to act as a support, scaffolding or skeleton for the annulus. When the band is secured in its pre-set configuration to the annulus, it does not only narrow the circumference of the dilated annulus, but it also re-shapes the annulus. The three-dimensional shape of the annulus is discussed in further detail hereinbelow.

In a preferred embodiment, the band in the pre-set configuration is substantially saddle-shaped. Within the context of the invention, “saddle-shaped” includes bands comprising two or more curves with the apex facing a first direction, and two or more curves facing a second direction, the second direction being opposed to the first direction. In a preferred embodiment, the saddle shaped band comprises two upward facing curves and two downwards facing curves. This pre-set configuration is particularly beneficial in that it defines the anatomical contours of a working annulus.

Other suitable shapes, contours or portions of the band allowing to reshape the annulus into a working configuration are envisaged including, but not limited to, an elongated member, a partial or complete ring, a partial or complete oval and the like. Angular shapes, contours or portions may be included, but curved or linear shapes are preferred to avoid tissue trauma and facilitating delivery of the device to the target site.

The band may be open or closed. The open band may be substantially linear, C-shaped, U-shaped, V-shaped, L-shaped and the like. The closed band may be substantially circular, oval, rectangular, D-shape and the like. Other shapes are envisaged, such as polygonal. The band may comprise one or more sections and each section may have the same or different shape.

Preferably, the pre-set configuration of the band is three-dimensional, as is the annulus.

Preferably, the device, in particular the band, comprises or consists of one or more plastics, rubber or metal materials. More preferably, said one or more plastics, rubber or metal materials are elastic or have elastic properties. For example, the band may consist of one or more elastic materials so as to be expandable and/or compressible from its pre-set configuration. Alternatively, the band may comprise one or more sections consisting of one or more elastic materials.

Examples of materials include plastics such as silicone-containing materials, rubbers, fabrics, metals such as shape-memory materials and alloys (in particular nitinol).

Preferably, the device comprises or consists of one or more bioabsorbable materials. Bioabsorbable materials are particularly advantageous in the context of the present invention, in that once the annuloplasty device is implanted, fibrous connective tissue will form around the device and will itself act as a natural support for the annulus. As the bioabsorbable materials are slowly absorbed, the annulus is maintained in its working configuration by the newly formed tissue.

Preferably, the device comprises a compressible and/or expandable structure. In this embodiment, the compression and/or expansion is achieved by mechanical means.

The band may comprise an expandable and/or compressible mesh or structure. For example, the band, mesh or structure may have a grid, diamond, serrated, crenelated, sinusoidal, spiral, helical and/or other patterns. The band may be integrally formed.

The band may comprise mechanical means such as hinged connections, elastic connections, springs, pivotable and or rotatable connections to achieve expansion and/or compression.

The device may comprise a tubular band, ring or crown comprising or consisting of any one or more of the materials, meshes or structures described above.

In an embodiment, the outer surface of the band comprises a protective cover, such as a layer of material and/or a coating. The cover may be provided to enhance the contact and interaction with the surrounding tissue, to promote fibrosis and tissue growth. The cover may comprise or consist of a mesh or a woven fabric.

The cover preferably comprises or consists of a flexible material, flexible enough to allow expansion and compression of the band. Examples of materials include polymers, such as polyester and polytetrafluoroethylene (PTF).

The protective cover may comprise one or more compounds to minimise the risk of thrombosis or inflammation. Examples of compounds include anti-coagulants, anti-inflammatory compounds, anti-bacterial compounds, anti-viral compounds, and the like. The protective cover may comprise one or more pro-healing compounds or compositions, i.e. compounds or compositions promoting healing.

The band according to the present invention could be anchored by means of sutures in a conventional manner. However, it is an object of the present invention to provide a device and a procedure which are simple and standardised. Hence, the device preferably comprises means for anchoring the band to the annulus.

The anchoring means may be mechanical anchoring means such as barbs, pegs, hooks, spikes and the like. The anchoring means may be either connected to, adhered to or glued to the band. Preferably integrally-formed with the band, to provide a safer device which is less resource intensive and simpler to manufacture. Using integrally-formed anchoring means also results in a device which is easier to implant, with fewer independently movable components.

Bioglues and bioadhesives are also envisaged within the context of the present invention, and the band, or its cover where applicable, may be coated with one or more adhesives instead of, or preferably, in addition to the mechanical anchoring means.

In a preferred embodiment, the device comprises means for automatically deploying the anchoring means. Preferably, the anchoring means automatically deploy and/or secure to the annulus upon release of the band from its expanded configuration. The anchoring means may be connected to the band so that, in the pre-set configuration of the band, the anchoring means are in their anchored configuration.

In an embodiment, the band may comprise anchoring means, which deploy and/or secure to the annulus by rotation of the band around its longitudinal axis (if the band is elongated) and/or by application of pressure (for example by pressing the anchoring means into the tissue of the annulus).

According to a second aspect of the present invention, there is provided a method for repairing a heart valve comprising the steps of implanting an annuloplasty device as described hereinabove to the heart valve annulus.

Preferably, the method comprises the step (a) of compressing the band to slidably fit in the lumen of a delivery catheter or sheath, so as to deliver the device to the target area.

Preferably, the method comprises the step (b) of expanding or deforming the band to a dimension equal or greater than a dimension of the heart valve annulus, so as to contact the anchoring sites on the dilated annulus.

Preferably, the method comprises the step (c) of releasing the expanded band into its pre-set configuration. Preferably, the method comprises the step of anchoring the band by releasing the expanded band into its pre-set configuration.

Preferably, the heart valve is the mitral valve, and the annulus is the anterior annulus, the posterior annulus, the lateral commissure and/or the medial commissure. Most annuloplasty procedures are directed at reducing the posterior annulus. However, the portions between the anterior and posterior annulus (the commissures) are also known to elongate over time and can be reshaped using a device according to the present invention.

Preferably, the heart valve is the tricuspid valve, and the annulus is the anterior annulus, the posterior annulus and/or the septal annulus.

According to a third aspect of the present invention, there is provided an annuloplasty device for restoring a heart valve annulus from a dysfunctional configuration to a working configuration, wherein the device comprises a support structure comprising a distal mouth, wherein the support structure is expandable from a pre-set configuration, wherein the pre-set configuration of the mouth corresponds to a working configuration of the annulus; and means for anchoring the mouth to the annulus.

In this aspect, the band is replaced by the mouth of the expandable (and retractable) support structure, and the pre-set configuration of the mouth of the support structure corresponds to a working configuration of the annulus. In other words, the band and/or the mouth is the part of the annuloplasty device which surrounds and interacts with the annulus to support the annulus in its working configuration.

In some embodiments, the mouth may be defined by the aperture of the annuloplasty device at a distal or proximal end thereof, for example by the outer periphery of the aperture.

The support structure fulfils the dual function of providing structural support to the device and of defining a mouth.

Structural support is advantageous in that it allows the mouth to return and remain in its pre-set configuration. In other words, it assists in the retraction of the mouth into the pre-set configuration during the implantation process and it minimises or prevents the deformation of the mouth once it is implanted on the annulus. The structural support may increase the inward force required to return the mouth to its pre-set configuration against the outward force exerted by the dilated annulus.

Structural support may for example be provided by mechanical means, the choice of material, shape and/or dimensions of the structural support. These features may be selected and adjusted to impart the intended support. For example, a material may be selected to provide the intended properties, including but not limited to expansion, retraction, resilience, rigidity and flexibility.

Preferably, the structural structure comprises a mesh structure, which may comprise a diamond-pattern. The diamond pattern is a simple structure allowing the control and adjustment of the expansion and retraction forces of the device, whilst providing structural rigidity to the device. The shape and dimensions of the diamonds and the pattern is not particularly limited. However, two rows have been shown to provide adequate support.

The structural support may comprise one or more resilient members, which may define substantially sinusoidal, crenelated or zigzag pattern. One or more members may be provided of the same or difference patterns.

The mouth has a pre-set configuration corresponding to a working configuration of the annulus, defined hereinabove in relation to the annuloplasty with a band. The mouth of the device may be defined by an aperture of the support structure (e.g. the distal aperture). The aperture may or may not comprise a band as described hereinabove in relation to the annuloplasty with a band.

Within the context of the present invention, it is envisaged to provide a device according to the first aspect comprising a structural support according to the third aspect. In other words, the device may comprise a band and a structural support.

The device comprises means for anchoring the mouth to the annulus. The anchoring means may be the same or different from the anchoring means described in relation to the annuloplasty with a band. Preferably, the anchoring means extend from the mouth of the support structure. The anchoring means may extend substantially longitudinally from the mouth of the support structure, or an angle inwards relative to the mouth of the support structure.

According to a fourth aspect of the present invention, there is provided an annuloplasty device for restoring a heart valve annulus from a dysfunctional configuration to a working configuration, wherein the device comprises a bridge structure having a first end and a second end; first means for anchoring the device to the annulus at the first end of the bridge structure, and second means for anchoring the device to the annulus at the first end of the bridge structure; wherein the device is expandable from a pre-set configuration, wherein the pre-set configuration of the device is configured to support the annulus in its working configuration.

According to a fifth aspect of the present invention, there is provided a method for repairing a heart valve comprising the steps of implanting a device comprising a support structure comprising a mouth, wherein the support structure is expandable from a pre-set configuration, wherein the pre-set configuration of the mouth corresponds to a working configuration of the annulus; and means for anchoring the mouth to the annulus.

The bridge structure may comprise or consist of a resilient material, such as a shape-memory material (e.g. nitinol). The anchoring means may be selected from the anchoring means described herein with respect to the first and third aspects.

In a preferred embodiment, the annuloplasty device comprises a resilient bridge structure comprising at least one anchor at each end thereof. The device may be folded for delivery through a sheath. The device may deploy upon exiting the sheath. The device may be expanded or stretched beyond its pre-set configuration. The device may be anchored to the annulus by means of the anchoring means, and released into its pre-set configuration.

In accordance to a fifth aspect of the present invention, there is provided a method for the transcatheter delivery of an annuloplasty device as described herein to a heart valve annulus, comprising the step of releasing an annuloplasty device as described hereinabove into its pre-set configuration.

The invention will be further described with reference to the drawings and figures, in which

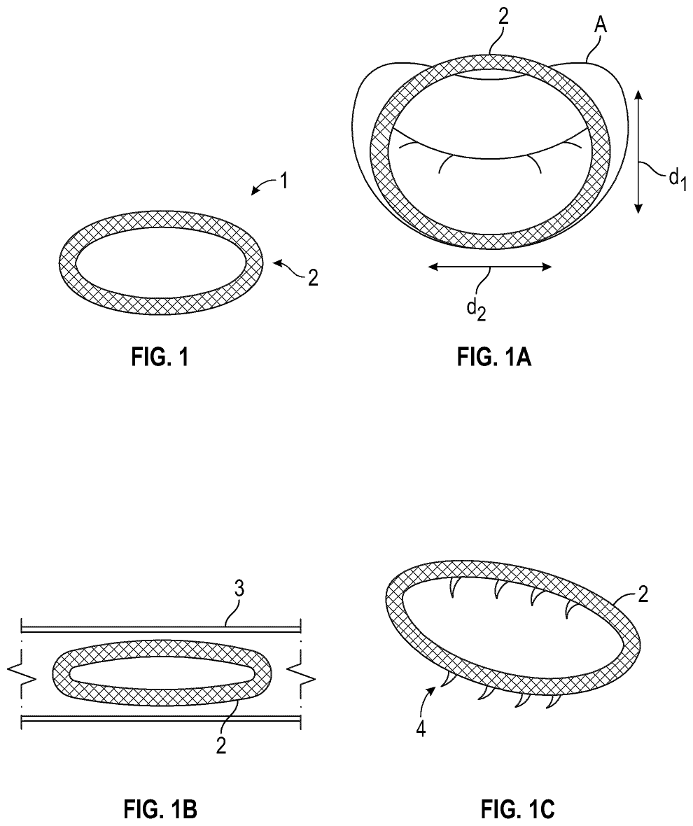

FIG. 1 is a schematic representation of an annuloplasty device according to the present invention, in its pre-set configuration;

FIG. 1a is a schematic representation of the annuloplasty device of FIG. 1 in an expanded configuration;

FIG. 1b is a schematic representation of the annuloplasty device of FIG. 1 in a compressed configuration;

FIG. 1c is a schematic representation of the annuloplasty device of FIG. 1 illustrating anchoring means;

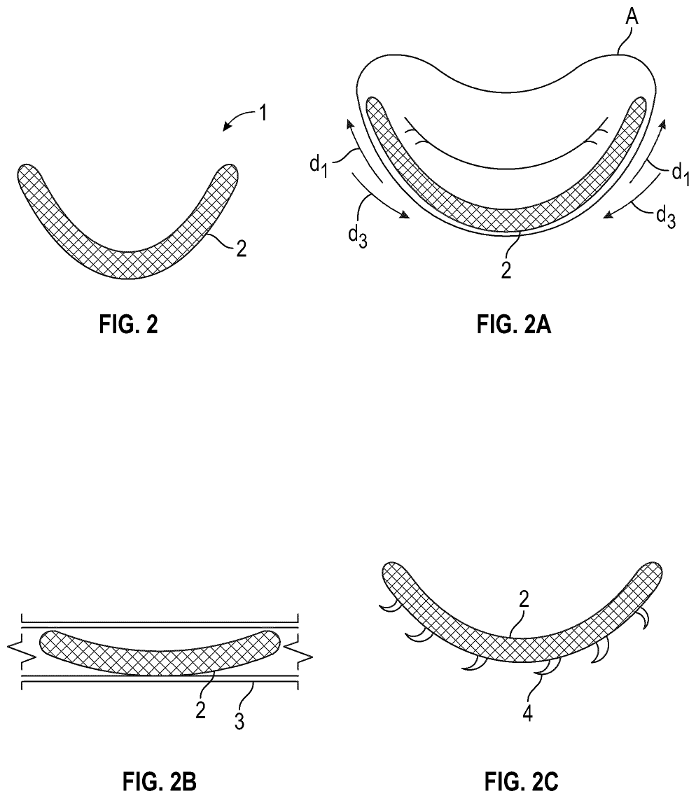

FIG. 2 is a schematic representation of an annuloplasty device according to the present invention, in its pre-set configuration;

FIG. 2a is a schematic representation of the annuloplasty device of FIG. 2 an expanded configuration;

FIG. 2b is a schematic representation of the annuloplasty device of FIG. 1 a compressed configuration;

FIG. 2c is a schematic representation of the annuloplasty device of FIG. 2 illustrating anchoring means;

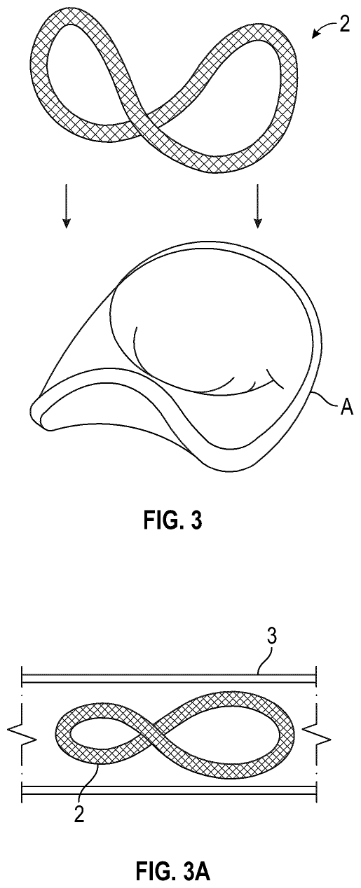

FIG. 3 is a schematic representation of an annuloplasty device according to the present invention, in its pre-set configuration;

FIG. 3a is a schematic representation of the annuloplasty device of FIG. 3 a compressed configuration;

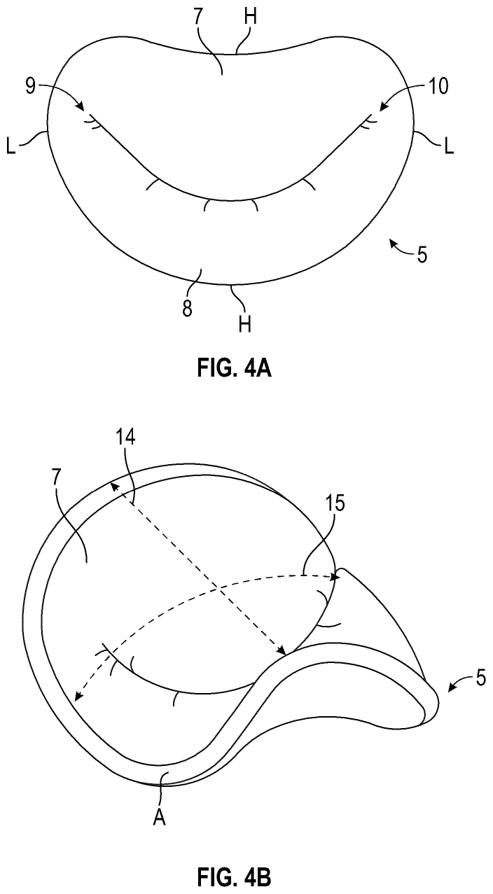

FIG. 4a is a schematic top representation of a working mitral valve;

FIG. 4b is a schematic representation of the three-dimensional shape of a working mitral valve;

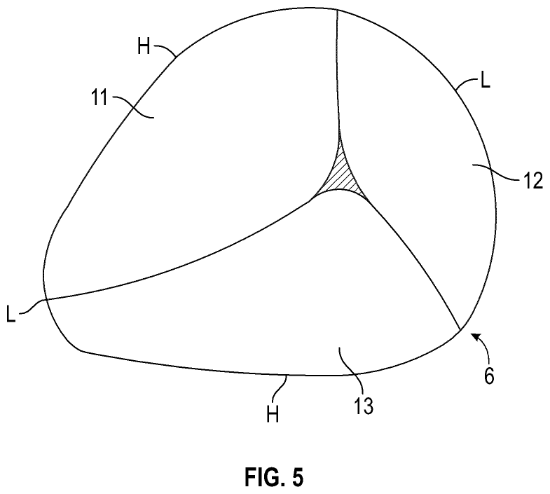

FIG. 5 is a schematic top representation of a working tricuspid valve;

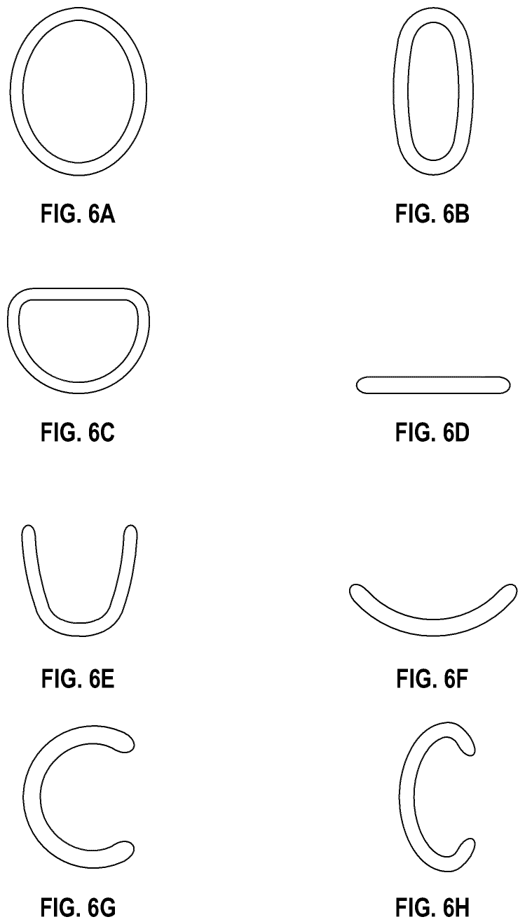

FIGS. 6a to 6h are schematic top representations of annuloplasty device shapes;

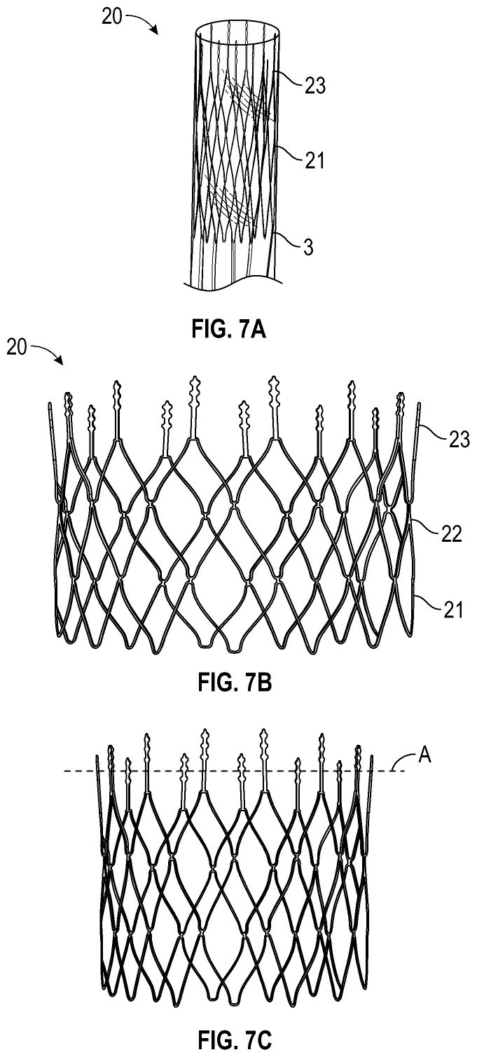

FIGS. 7a to 7c are schematic representations of an annuloplasty device according to the present invention, its delivery configuration, in its expanded configuration and in its pre-set configuration, respectively; and

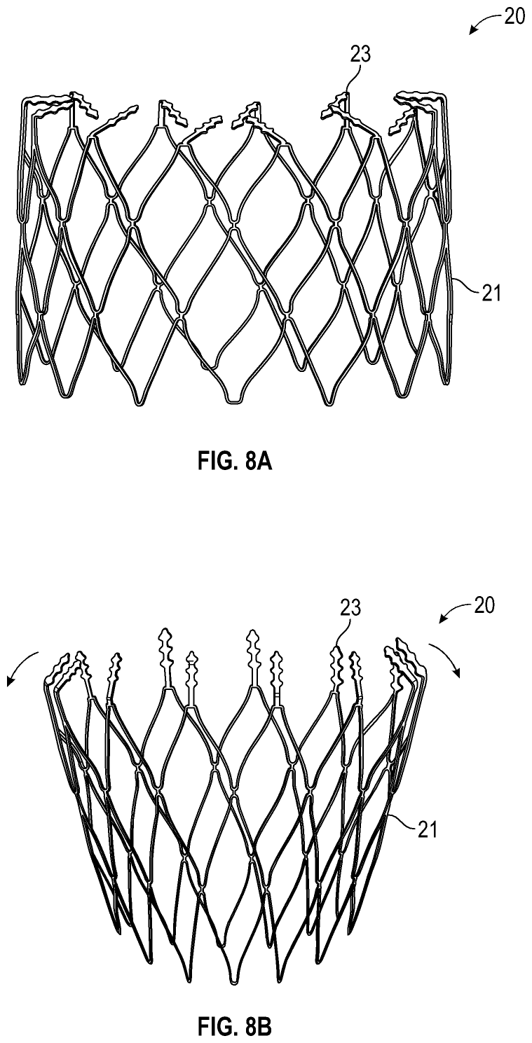

FIGS. 8a and 8b shows an annuloplasty device according to the present invention which anchoring means extending inwards.

The embodiments described herein are provided as exemplary and non-limiting embodiments of the present invention.

With reference to FIG. 1, there is illustrated an annuloplasty device 1 for restoring a heart valve annulus A from a dysfunctional configuration to a working configuration, wherein the device 1 comprises a band 2 which is expandable from a pre-set configuration, wherein the pre-set configuration of the band 2 corresponds to a working configuration of the annulus A.

In the embodiment illustrated in FIG. 1, the annuloplasty device 1 comprises a closed band 2, which is tubular and formed of a mesh. The mesh structure is expandable as illustrated in FIG. 1a, and may be compressible as illustrated in FIG. 1b. The structure may have a pattern, such as a grid, diamond, serrated, crenelated, sinusoidal, spiral, helical and/or other patterns. Alternatively, the band 2 may be a solid band. The band 2 may comprise an elastic material or a shape-memory alloy, such as nitinol.

The band 2 is shown in an exemplary substantially oval pre-set configuration in FIG. 1, which corresponds to a working configuration of the annulus A. In FIG. 1a, the band 2 is illustrated in an expanded anchoring configuration, in which the dimensions of the band 2 can be expanded in directions d1 and/or d2 to reach the attachment sites of the annulus A. In FIG. 1b, the band 2 is illustrated in a compressed delivery configuration, in which the band 2 is compressed into a delivery catheter 3, so that the band 2 can be pushed along the inner lumen of the delivery catheter 3. In FIG. 1c, the band 2 comprises exemplary anchoring means 4, in the form of pegs. The anchoring means 4 are connected to the band 2, partially or completely along the band 2.

In the embodiment illustrated in FIG. 2, the annuloplasty device 1 comprises an open band 2, which is tubular and formed of a mesh. The band in FIG. 2 is shown in an exemplary curved pre-set configuration, which corresponds to a working configuration of the annulus A.

In FIG. 2a, the band 2 is illustrated in an expanded anchoring configuration, in which the dimensions of the band 2 can be expanded in directions d1. Upon release of the expanded band 2, the band 2 retracts into its pre-set configuration in direction d3.

In FIG. 2b, the band 2 is illustrated in a compressed delivery configuration, in which the band 2 is compressed into a delivery catheter 3, so that the band 2 can be pushed along the inner lumen of the delivery catheter 3. The band 2 may be compressed to that its radial dimension is decreased, or the band 2 may be folded upon itself to decrease its overall longitudinal dimension. In FIG. 2c, the band 2 comprises exemplary anchoring means 4, in the form of hooks. The anchoring means 4 are connected to the band 2, partially or completely along the band 2.

In the preferred embodiment illustrated in FIG. 3, the annuloplasty device 1 comprises a substantially saddle-shaped band 2. The shape of the band 2 in its pre-set configuration corresponds to and restores the annulus A to its working configuration. The band 2 may be expanded (not shown) in any direction owing to the material and/or the structure of the band 2, as described hereinabove. In the expanded configuration, the band 2 reaches and anchors to the attachment sites of the degenerated or dilated annulus A. Upon release of the band 2 from its expanded configuration, the band 2 reverts into its pre-set configuration, thereby reshaping the annulus A to its working configuration.

FIGS. 6a to 6h are top views of exemplary pre-set working configurations for the annuloplasty device 1. The device 1 is preferably three-dimensionally shaped to correspond to the three-dimensional shape of a working annulus and/or to restore a dysfunctional annulus to a working configuration. The device 1 may have a closed shape (as shown in FIGS. 61 to 6c) to surround the valve. Alternatively, the device 1 may have an opened shape (as shown in FIGS. 6d to 6h) to partially surround and restore the valve.

FIGS. 7a to 7c are provided to illustrate additional or alternative features of the annuloplasty device as described hereinabove. An annuloplasty device 20 is provided with a support structure 21 comprising a distal mouth 22, wherein the support structure 21 is expandable from a pre-set configuration (FIG. 7b), wherein the pre-set configuration of the mouth 22 corresponds to a working configuration of the annulus; and means 23 for anchoring the mouth to the annulus.

In FIG. 7a, the annuloplasty device 20 is illustrated in its delivery configuration, folded so as to fit in a delivery catheter 3. In this embodiment, the annuloplasty device 20 is its folded configuration has smaller outer dimensions than those of the pre-set configuration.

In FIG. 7b, the annuloplasty device 20 is illustrated in its expanded configuration. During the implantation process, the support structure 21 is expanded (by means of a delivery device—not shown), so that the dimensions of the mouth 22 are greater than those of the annulus and greater than those of the mouth 22 in the pre-set configuration.

Once suitably positioned, the device 20 is released so that the anchoring means 23 (in the present embodiment comprising barbs) grab and anchor to or into the tissue surrounding the annulus. The mouth 22 reverts to its pre-set configuration as shown in FIG. 7c, so that the annulus is reverted to a working configuration.

In FIGS. 7a to 7c, the anchoring means 23 extend longitudinally from the support structure 21. However, the anchoring means 23 may extend inwardly relative to the mouth and/or support structure as shown in FIGS. 8a and 8b. This inward angle has the advantage of improving the grabbing of and anchoring to the patient's tissues.

In situ, the support structure 21 may extend away from the annulus or may be located partially or completely through the annulus.

A method for repairing a heart valve will now be outlined, using device 1 from FIG. 3.

The device 1 is detachably connected to a delivery device (not shown). For example, the distal detachment may be controlled proximally. Within the context of the present invention, “proximal” side refers to the side closest to the medical practitioner and outside the patient; “distal” refers to the side closest to the target annulus.

The device 3 is compressed (for example as illustrated in FIG. 3a) to fit into the inner lumen of a delivery catheter 3. The band 2 in its compressed configuration is compressed so as to decrease its radial dimension and slidably fit in the delivery catheter 3. The device 1 is pushed by the delivery device along the length of the catheter 3. The distal end of the catheter 3 is positioned adjacent the pathologically degenerated annulus A.

As the device 1 exits the delivery catheter 3, the device 1 naturally reverts to its pre-set configuration. The device 1 is positioned so that its contours corresponds to the general contours of the annulus (as shown in FIG. 3).

For a better understanding of the present invention, reference is made to FIGS. 4a, 4b and 5, illustrating the anatomy of the mitral valve 5 and the tricuspid valve 6.

The mitral valve 5 comprises an anterior leaflet 7, a posterior leaflet 8, a lateral leaflet 9 and a medial leaflet 10. The tricuspid valve 6 comprises an anterior leaflet 11, a posterior leaflet 12 and a septal leaflet 13.

The mitral valve 5 and tricuspid valve 6 do not have a planar configuration, but are curved and saddle-shaped. FIGS. 4a and 5 show the relatively high areas H and the relatively low areas L. The device 1 according to the present invention preferably does not have a substantially planar configuration. Instead, the device 1 is curved, and preferably saddle-shaped, so as to follow the natural configuration of the annulus A.

The device 1 is positioned adjacent the annulus A so that the device 1 is above the area of the annulus to be reshaped. This area may be the whole perimeter of the annulus A, or one or more portions of the annulus A. For example, a saddle-shaped device 1 may be used to reshape the whole annulus, or an open band device 1 may be used to reshape e.g. the posterior leaflet 8, 12 only.

From this position, and using the delivery device, the device 1 is expanded from its pre-set configuration so that the anchoring means 4 reach the attachment sites of the degenerated annulus. The attachment sites are preferably such that the configuration they outline has greater dimensions than those of the pre-set configuration of the device 1. Therefore, in order to grasp and anchor to the attachment sites of the annulus A, the device 1 is expanded.

Once the device 1 is suitably expanded, the anchoring means 4 are anchored to the attachment sites of the annulus A. The device 1 may be sutured to the attachment sites. However, it is preferred to avoid such an intricate and irreversible procedure by providing the device 1 with anchoring means. The anchoring means 4 may be hooks or pegs or other means which may be pushed into the tissue of the annulus A.

In an embodiment, the anchoring means 4 are pressed into the tissue of the annulus A upon release of the annuloplasty device 1, owing to the device 1 reverting to its pre-set configuration. Alternatively or additionally, the delivery device may be used to press on the anchoring means 4, and/or the arms may be used to manipulate and/or rotate the band 2 so that the anchoring means 4 (for example hooks) grasp and secure to the annulus A.

It is also envisaged that the annuloplasty device 1 is anchored to the “back” of the annulus, for example on the ventricular side of the annulus. In this embodiment, the delivery sheath 3 is pushed through the annulus, until the distal end of the sheath 3 is positioned beyond the annulus. The device 1 is pushed forward, and deploys upon exiting the delivery sheath 3. The device 1 is expanded beyond its pre-set configuration and suitably positioned to catch the annulus. The device 1 is anchored to the back of the annulus and released into its pre-set configuration.

It may be advantageous to provide structural support to the back of the annulus, if the back of the annulus is better defined, has better tissue integrity and provides more suitable anchoring sites. In addition, the anchoring step may be improved by using the pulling (in the proximal to distal direction) the annuloplasty device 1 to provide greater anchoring force.

A problem arising from conventional annuloplasty rings is that their implantation is difficult to reverse. In other words, the rings cannot be easily removed and repositioned in the event of an unsuccessful implantation. In the present invention, anchoring means 4 secured to the annulus by pressure from the annuloplasty device 1 (owing to its pre-configuration) may be used, so that should the device 1 require repositioning, the expansion of the device 1 would release the anchoring means 4. The device 1 can be repositioned with minimal trauma to the annulus A.

Although the present invention has been described within the context of heart valves and in particular of annuloplasty procedures, it is envisaged that it could have other advantageous implementations involving the reshaping of an anatomical structure.

The present invention is particularly beneficial in the context of transcatheter procedures and/or in the cardiovascular field. For example, in transcatheter procedures, access points are formed through tissues and/or blood vessels. The device may be used to restore blood vessels to their working configuration by partially or completely closing the access point(s) so as to minimise or prevent blood flow to the outside of the blood vessels.

The present invention may be used in the context of an atrial septal defect (ASD), which is a hole in the atrial septum. The ASD may be congenital, or may be the desired result of a medical procedure intended to relieve the pressure between the two atrial chambers of the heart. The device may be used to partially or completely close the hole so as to minimise or prevent blood flow between the atrial chambers.

Thus, the present invention provides an annuloplasty device for preventing further deterioration of the annulus', for improving or restoring the annulus' function and/or for palliative support. The device is simple and standardisable. The development and implantation of the device in patients becomes less resource-intensive when compared to conventional devices. The present annuloplasty device can be delivered and implanted using a transcatheter method, which is minimally invasive. The device is easily secured to a dysfunctional annulus in order to restore it to a healthy working configuration. This requires minimal manipulation, positioning and adjustment in situ. The medical practitioners require less extensive training as would be required to implant conventional annuloplasty devices. Importantly, the annuloplasty device according to the present invention restore the shape, in particular the three-dimensional shape, of the annulus instead of simply reducing the opening of the valve.

1. An annuloplasty device for restoring a heart valve annulus from a dysfunctional configuration to a working configuration, wherein the device comprises a band which is expandable and compressible from a pre-set configuration, wherein the pre-set configuration of the band corresponds to the working configuration of the annulus.

2. (canceled)

3. The annuloplasty device according to claim 1, wherein the band, in its compressed configuration, slidably fits through a lumen of a delivery catheter.

4. The annuloplasty device according to claim 3, wherein the dimensions of the band in the expanded configuration are equal or greater than the dimensions of the dysfunctional annulus.

5. The annuloplasty device according to claim 1, wherein the band in the pre-set configuration is substantially rigid.

6. The annuloplasty device according to claim 5, wherein the band in the pre-set configuration is substantially saddle-shaped.

7-10. (canceled)

11. The annuloplasty device according to claim 1, comprising:
an anchoring means for anchoring the band to the annulus, the anchoring means is integrated with the band; and
a means for automatically deploying the anchoring means.

12-14. (canceled)

15. The annuloplasty device according to claim 12, comprising a means for forcing the band into its pre-set configuration.

16. (canceled)

17. A method for repairing a heart valve annulus in a patient in need thereof, the method comprising implanting a annuloplasty device for restoring a heart valve annulus from a dysfunctional configuration to a working configuration, wherein the device comprises a band which is expandable and compressible from a pre-set configuration, wherein the pre-set configuration of the band corresponds to the working configuration of the annulus, the heart valve annulus, implanting the device includes:
compressing the band to deliver the device to the annulus through a delivery catheter;
expanding the band to a circumference equal or greater than the circumference of the annulus; and
releasing the band into the pre-set configuration.

18-20. (canceled)

21. The method according to claim 17, wherein the heart valve is the mitral valve, and the annulus is selected from a group of: the anterior annulus, the posterior annulus, the lateral commissure, and the medial commissure.

22. The method according to claim 17, wherein the heart valve is the tricuspid valve, and the annulus is selected from a group of: the anterior annulus, the posterior annulus, and the septal annulus.

23. An annuloplasty device for restoring a heart valve annulus from a dysfunctional configuration to a working configuration, the device comprises:
a support structure comprising a mouth, wherein the support structure is expandable from a pre-set configuration, wherein the pre-set configuration of the mouth corresponds to the working configuration of the annulus; and
an anchoring means for anchoring the mouth to the annulus.

24. The annuloplasty device according to claim 23, wherein
the support structure comprises an expandable mesh having a diamond-pattern.

25. The annuloplasty device according to claim 24, wherein the support structure is selected from among a substantially sinusoidal, crenelated and zigzag pattern.

26. The annuloplasty device according to claim 23, wherein the anchoring means extend from the mouth of the support structure.

27. (canceled)

28. The annuloplasty device according to claim 26, wherein the anchoring means extend inwards relative to the mouth of the support structure.

29. (canceled)

30. The device according to claim 28, wherein the device is configured to be implanted and the support structure is compressed to deliver the device to the heart valve annulus through a delivery sheath and the support structure is expanded to a circumference equal or greater than the circumference of the heart valve annulus and the support structure is released into the pre-set configuration.

31-32. (canceled)

33. The device according to claim 30, wherein the heart valve is the mitral valve, and the annulus is selected from a group of: the anterior annulus, the posterior annulus, the lateral commissure, and the medial commissure.

34. The device according to claim 30, wherein the heart valve is the tricuspid valve, and the annulus is the anterior annulus, the posterior annulus, and the septal annulus.

35. An annuloplasty device for restoring a heart valve annulus from a dysfunctional configuration to a working configuration, the device comprising:
a bridge structure having a first end and a second end;
first means for anchoring the device to the annulus at the first end of the bridge structure; and
second means for anchoring the device to the annulus at the first end of the bridge structure;
wherein the device is expandable from a pre-set configuration, wherein the pre-set configuration of the device is configured to support the annulus in its working configuration.

36. (canceled)
